(19) United States
(12) Patent Application Publication
(10) Pub. No.: US 2008/0200780 A1
(43) Pub. Date: Aug. 21, 2008

(54) OPTICAL MEASUREMENT OF CELLULAR ENERGETICS

(76) Inventors: Kenneth A. Schenkman, Seattle, WA (US); Lorilee S.L. Arakaki, Bainbridge Island, WA (US); Wayne A. Ciesielski, Seattle, WA (US); Eric O. Feigl, Seattle, WA (US); David H. Burns, Point Claire (CA)

Correspondence Address: SCHWEGMAN, LUNDBERG & WOESSNER, P.A., P.O. BOX 2938, MINNEAPOLIS, MN, 55402, US

(21) Appl. No.: 11/947,242

(22) Filed: Nov. 29, 2007

Related U.S. Application Data
(63) Continuation-in-part of application No. 11/382,879, filed on May 11, 2006.

(51) Int. Cl. A61B 5/00

(52) U.S. Cl. 600/310

(57) ABSTRACT

This document discloses, among other things, systems and methods for measuring a level of myoglobin, hemoglobin, and cytochrome using an optical probe coupled to a spectrometer. Multivariate analysis of the spectral data yields quantifiable cellular and mitochondrial characteristics.

### CLAIM OF PRIORITY

This application is a continuation-in-part of Schenkman, U.S. patent application Ser. No. 11/382,879, entitled “OPTICAL MEASUREMENT OF MITOCHONDRIAL FUNCTION IN BLOOD PERFUSED TISSUE,” filed on May 11, 2006, (Attorney Docket No. 2082.007US1), and is incorporated herein by reference.

### STATEMENT OF GOVERNMENT RIGHTS

This invention was made with Government support under Grant Number R01AR041928 awarded by the National Institutes of Health (NIH). The Government has certain rights in this invention.

### TECHNICAL FIELD

This document pertains generally to measurement of tissue characteristics, and more particularly, but not by way of limitation, to optical measurement of cellular energetics.

### BACKGROUND

Delivery of oxygen to the tissues and organs of the body is important for life. The mitochondria, which are the energy producing units of the cells in the body, require oxygen for energy production. Inadequate oxygen delivery to tissue may result in organ dysfunction and even death.

Each year, approximately 750,000 patients present to hospitals in the United States with severe sepsis, and 215,000 of these patients die from their illness. An estimated two million inpatient hospital visits are made each year in this country due to traumatic injuries, and of this group, there are 150,000 deaths.

Currently, clinical measurements of tissue oxygenation rely on indirect measures. Some indicators of tissue oxygenation include blood pressure and blood gases. Indirect measurements are inadequate.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a spectrometer configuration.

FIG. 2 illustrates a view of a probe.

FIG. 3 illustrates a portable device.

FIG. 4 illustrates a flow chart of a method.

FIG. 5 illustrates a flow chart of a method including an iterative process.

### DETAILED DESCRIPTION

The task of determining cellular energetics entails determining the constituents of a sample tissue. One example of the present subject matter detects oxygen changes at the cellular or subcellular level by illuminating the sample with light and analyzing a reflected composite output signal. Analysis using multivariate curve resolution (MCR) can be used to determine cellular energetics.

The present subject matter provides a method to determine at least one in vivo cellular energetic parameter, including cellular and mitochondrial state of tissue, such as, for example, the heart during or after cardiac surgery, or in skeletal muscle in a range of medical conditions. Mitochondrial characteristics can be determined in tissues other than muscle as well.

The cellular and mitochondrial oxygenation state can be measured optically using a number of parameters including, for example, intracellular oxygenation and redox state. Reflectance optical spectroscopy provides a non-invasive measure of absorbance in tissue, thus allowing quantitative determination of intracellular oxygenation, blood oxygenation, and redox states of mitochondrial cytochromes.

The present subject matter includes a cellular and mitochondrial oxygenation monitor which may be useful in physiology, pathology, and in applied clinical areas such as intensive care, cardiac surgery (e.g., during by-pass surgery), and other surgeries. Such measurements may help in the study of the control of oxidative metabolism in the muscle cell. In vivo, real-time measurements of cellular and mitochondrial function may be useful for clinical monitoring in critical care or during conditions of suspended animation. A portable device embodying the present subject matter may provide means for improved control and monitoring of medical and surgical procedures. Such a device, when used during resuscitation, may help to decrease mortality in critically ill and injured patients.

Light from tissue (especially in visible and near infrared regions) is absorbed by hemoglobin, myoglobin and the cytochromes, among other absorbing components. Myoglobin is found in skeletal and cardiac muscle and primarily functions as an oxygen storage or transport molecule. Within the cell, myoglobin carries oxygen generally from the capillary side of the muscle cells to the mitochondria. The absorbance spectrum of myoglobin changes as a function of oxygen binding and differences in the oxygenated and deoxygenated state of the molecule are measurable in both the visible and near-infrared spectral regions.

Hemoglobin, on the other hand, which also has similar absorbance changes with oxygenation, carries oxygen in the blood from the lungs throughout the body. A measure of hemoglobin oxygen binding is indicative of oxygen supply.

Myoglobin and hemoglobin saturation are defined as the percentage of myoglobin or hemoglobin that is bound to oxygen.

Tissue also includes cytochromes in the mitochondria. Cytochromes are part of the electron transport chain and mitochondria generate the energy to keep cells alive. Electrons are carried in the fuel source provided to the mitochondria, and travel down a chain of enzymes and eventually, reduce oxygen to water. The redox states of cytochromes (oxidized vs. reduced) create a spectral shift that can be identified.

Two particular cytochromes, cytochrome c and cytochrome oxidase, are recognized as the last two enzymes of the electron transport chain. A spectral change occurs with a change in the redox state of these enzymes when electrons pass through this chain.

Cytochrome redox is defined as the percentage of total cytochrome (c or oxidase or other) that is in the oxidized state.

Analysis at the myoglobin level can reveal the oxygen actually within the cell and analysis at the mitochondria level can reveal how the oxygen is being used.

In one example, the present subject matter provides a measure of oxygenation at the cellular level and mitochondrial function within tissues.

Current medical monitoring of oxygen availability is limited to assessment of arterial blood oxygenation, either by pulse oximetry (for detecting the percentage of hemoglobin saturated with oxygen) or by intermittent blood gas sampling. Pulse oximetry and intermittent blood gas sampling can only measure oxygen that is being circulated in the bloodstream.

In pulse oximetry, measurements are typically taken from a sensor placed over the toe, finger or ear lobe. Two or more discrete wavelengths of light (one red and one infrared) are shined through the finger or other appendage to a photo-detector on the other side. Some of that light is absorbed by the presence of intervening blood and the result is displayed as a percent of oxygen saturation.

The present subject matter can provide information about tissue oxygenation that is more clinically relevant than pulse oximetry. Intracellular oxygen levels in muscle tissue indicate the balance between oxygen supply and utilization. Cytochrome reduction-oxidation (redox) states reflect the rates of energy production by the mitochondria. These measurements can be combined to monitor the internal workings of cells, namely, where oxygen is consumed and energy is produced.

The present subject matter provides direct measurements of cellular and mitochondrial function in the human heart, for example, during cardiac surgery, thus revealing how well the heart is being protected from damage during bypass, and can be used to assess skeletal muscle energetic states as well. In addition, the present subject matter may facilitate identification of optimal parameters for cardioplegia administration during heart surgery and other clinical therapies.

In one example of the present subject matter, an optical probe is used to directly assess the heart and quantitatively determine myoglobin oxygen saturation in cardiac tissue, which leads directly to measurements of intracellular oxygenation. The present subject matter also allows quantitative measurements of the redox states of cytochrome c and cytochrome oxidase in the mitochondria.

In one example of the present subject matter, an optical probe is used to directly assess skeletal muscle in the body and quantitatively determine myoglobin oxygen saturation in that muscle tissue, which leads directly to measurements of intracellular oxygenation. The skeletal muscle can include an arm, a leg, or other muscle. The present subject matter also allows quantitative measurements of the redox states of cytochrome c and cytochrome oxidase in the mitochondria.

In addition to cytochrome c and cytochrome oxidase, the present subject matter can be used to quantify cytochrome b as well as other cytochromes or other optically active species in a sample.

An example of the present system monitors a wide range of wavelengths of light (typically about 200 wavelengths) in the visible and near infrared region (NIR), thus distinguishing oxygen binding to myoglobin, an intracellular protein, from oxygen binding to hemoglobin in blood. This distinction is not possible with existing spectrometer systems that measure only a small number of wavelengths.

In addition, the present subject matter can simultaneously measure the redox state (or activity), of the cytochromes in the mitochondria (mitochondria are the energy producing “power houses” of the cell), thus determining the energy state of the cells.

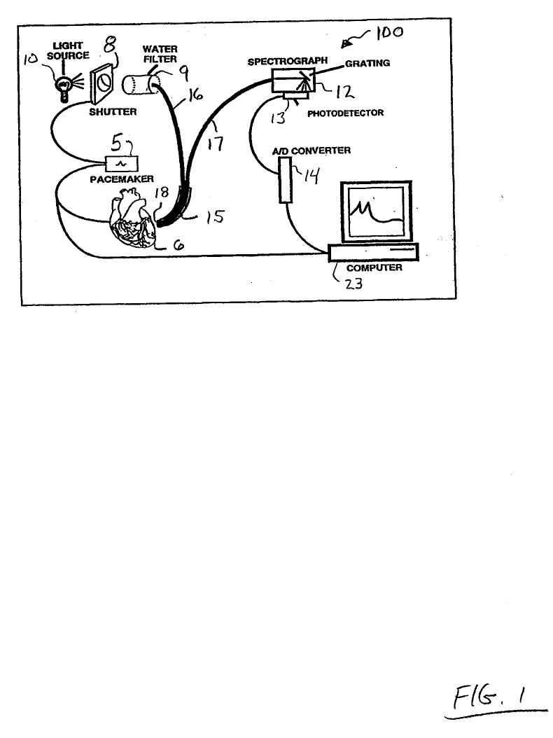

FIG. 1 illustrates system 100 for obtaining spectra using a fiber optic-based spectrophotometer for non-invasive measurement of cytochrome oxidation, hemoglobin saturation, and myoglobin saturation in muscle tissue in vivo.

By way of overview, system 100 includes detector 12 and fiber-optic probe 15. Light from source 10 is conveyed to probe 15, and then to detector 12. In one example, detector 12 includes a fiber-optic spectrophotometer having a grating and a photodetector, such as a photodiode array or a charge-coupled device (CCD) camera. Probe 15 provides a signal based on light source 10 and includes a bifurcated fiber optic element. Probe 15 is optically coupled to an input bundle 16 (illuminating fibers) and an output bundle 17 (detector fibers). In one example, bundle 16 and bundle 17 include a plurality of optically conductive fibers (such as glass fibers). Light is focused into input bundle 16 to illuminate the tissue and the reflectance signal, in the form of light received from the tissue, is conveyed to detector 12.

In one example, system 100 includes a broadband white light source to illuminate muscle tissue and detect color (spectral) changes in the reflected light returned to a spectrometer. Light source 10 can include a light emitting diode (LED), a QTH (quartz, tungsten-halogen) lamp, or other light source to provide visible light as well as near infra red energy. Electromagnetic energy in other ranges, including ultraviolet light, can also be used with the present system.

In one example, source 10 is pulsed to allow for gated data collection. In FIG. 1, shutter 8 can modulate the light during spectral acquisition. Modulating the light can also avoid excessive heating of tissue and sample damage caused by continuous illumination.

Shutter 8 can include a mechanical shutter, an electrical shutter, or an electro-optical shutter. Selective data collection can be triggered or timed by a selected event, for example a physiological event. For example, data collection from cardiac muscle can be triggered in in vivo measurements by the cardiac cycle, the respiratory cycle or both. In the figure, shutter 8 is synchronized with, for example, pacemaker 5 coupled to heart 6.

Filter 9, in the figure, removes mid-infrared wavelengths (heat) and can reduce tissue damage. In various examples, filter 9 includes a one-inch water filter (and possibly a water cooler), a dielectric mirror, or other apparatus to filter out a particular wavelength or range of wavelengths.

Detector 12 includes a spectrometer to generate a reflectance spectrum for a plurality of wavelengths. Various types of spectrometers are contemplated including those having a sensor and a prism, diffraction grating, or slit. The prism (or grating, or slit) can be stationary or swept and the sensor can be stationary or swept.

The reflected or backscattered light is focused onto sensor 13. Sensor 13 can include at least one of a photodetector, a photomultiplier tube, a photodiode, and a charge-coupled device (CCD). In one example, the elements of sensor 13 are arranged in an array.

In one example, sensor 13 includes a 512-pixel diode array photodetector. An electrical signal from sensor 13 is provided to analog-to-digital converter 14 and the resultant digital data is sent to computer 23 for storage and processing.

Detector 12, in one example, includes a slit and a diffraction grating coupled to photodiode array sensor 13, thus providing optical detection as a function of wavelength.

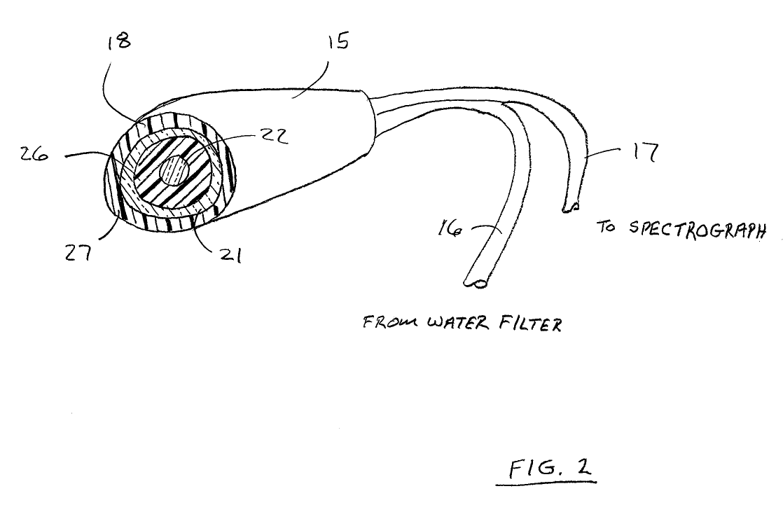

Probe 15 functions as an optical receiver for receiving an optical signal from blood-perfused tissue and can also be viewed as a source for providing spectral data. An example of probe 15 configured for reflectance measurement is illustrated in FIG. 2.

In the figure, probe 15 includes a concentric bull's-eye arrangement of optical fibers. Distal end 18 includes fiber bundle 22 and fiber bundle 21. Fiber bundle 22 is shown as an inner core and fiber bundle 21 is shown as an outer ring separated by insulation 26 and sheathed in outer insulation 27. Light from source 10 is delivered to a probe 15 by input bundle 16 and via ring shaped fiber bundle 21 and light reflected from the sample is received by center core, fiber bundle 22 and conducted to detector 12 by output bundle 17.

The radial distance between bundle 21 and bundle 22 is related to the depth of penetration of the optical signal, and thus, the region under measurement. In one example, the distance between input fiber bundle 21 and output fiber bundle 22 is adjustable and is selected to determine the depth of tissue sampling which is also a function of the wavelength illuminating a sample. The distance is selected to avoid mere sampling of superficial elements of tissue. Generally, the sampling depth increases with increased spacing between the illuminating and detector fibers. The distance is also adjusted to maintain a useful signal level returned to the detector fibers. The signal level generally decreases with increased spacing.

In one example, the source to detector separation is approximately 3 to 4 times the average depth of penetration of light into tissue, and thus setting the distance between the two sets of fibers between about 1 mm to about 3 mm provides an average penetration of about 0.25 mm to about 1.0 mm, respectively. In addition, the use of a contacting probe reduces the surface specular reflection of light contributing to the detected signal.

A variety of spacing (distance) dimensions and sampling depths are contemplated. For measurements in the visible wavelength region, the distance between fibers can be about 1 mm, or several mm, corresponding to an average sampling depth of approximately 0.25 mm or larger. For measurement in the near infra-red wavelength region, the distance between fibers can be about 3 mm or larger, corresponding to an average sampling depth of about 1.0 mm or more. The distance between the fiber bundles can be several millimeters for transcutaneous measurement of muscle. For some applications, the distance between the source and the detector bundle can be 1.6 cm, however, other dimensions, including a space of several centimeters, can also be used. In general, the signal-to-noise ratio will decrease with increased distance between the fiber bundles.

Other arrangements of illuminating and detector fibers at distal end 18 can be used. For example, a checkerboard arrangement of fibers, which maintains the desired distance between illuminating and detector fibers, can be employed. In one example, end 18 is configured with spaced strips of illuminating and detecting fibers. In one example, an input bundle is located in one radial section of the probe end and an output bundle is located in another radial section of the probe end. In addition, the input bundle and output bundle may be located on opposite sides of a tissue sample and thus, the system is sensitive to transmitted light rather than reflected light. In one example, the arrangement of input and output fiber bundles in probe 15 is reversed from that which is illustrated. In one example, probe 15 includes separate input and output optical fiber bundles.

Probe 15, in one example, includes fiber optic bundles held in a desired configuration to achieve a desired spacing and alignment between illuminating and detector fibers. For example, the fiber bundles can be inserted into an appropriately fabricated holder. The holder can be made of any inert, preferably non-toxic material, for example, metal, polymer material or plastic. End 18 of probe 15 is polished to obtain a highly smoothed surface, in which the fiber ends are substantially perpendicular to the plane of the distal end face. In one example, a mirrored surface is in contact with the tissue rather than the fibers themselves. A probe may be held in direct contact with an arm or leg to generate data concerning a muscle or it may be held at a distance from the skin. A catheter may be used to bring an optical probe to a position for measuring optical data for cardiac tissue. In one example, other structures or methods are used to receive optical data.

In one example, probe 15 is configured for human use and has no metallic parts to ensure that patients are electrically isolated from the spectrometer. In one example, probe 15 is configured to withstand repeated sterilizations in an autoclave.

With respect to FIG. 1, computer 23 includes an input interface for coupling to an A/D converter (such as converter 14) to receive measured data. In the figure, computer 23 is shown to include a keyboard having a plurality of user operable switches and a display. Various components can be included in computer 23, such as a mouse, trackball, touchpad, printer, an interface, and other elements configured for controlling or monitoring components of system 100, including, for example, shutter 8 and light source 10. Computer 23 can include a wired or wireless interface to couple with a communication network. Computer 23 is also configured to generate results and present data.

Computer 23 includes a processor configured to execute instructions for implementing an algorithm described elsewhere in this document. The instructions, algorithm, and data, can be stored in memory or received from an input. The memory, in various examples, includes a volatile or non-volatile memory or storage device.

In one example, computer 23 can be viewed as a data receiver for receiving spectral data based on blood-perfused tissue. A memory device of computer 23 stores the data and other spectra and a processor of computer 23 is configured to execute instructions to generate a mitochondrial characteristic using the spectra and the data.

The present subject matter can be employed for invasive or non-invasive measurement of muscle or other tissue. As used herein the term non-invasive includes measurements that inflict no damage to biological tissue, yet which may require contact with biological tissue. Invasive or minimally invasive measurement of tissue can include, for example those that may employ a trans-illumination needle probe inserted into the muscle tissue. An exemplary needle probe configuration includes two needle probes which are spaced apart, one of which carries an illuminating fiber and the other of which carries a detecting fiber. A transmission spectrum of the tissue between the two needle ends can be obtained with such a probe. One example includes both transmitting and detecting fibers in the same needle probe. In various examples, the present subject matter includes contacting or non-contacting probes. A variety of methods for contacting the fiber optic probe with a tissue sample (either in vivo or in vitro) can be employed. For example, cardiac muscle measurements can be obtained by direct contact with the heart muscle during surgery or indirectly by minimally invasive techniques, for example, via catheter insertion of the probe or via insertion of the probe by trans-esophageal methods as used in trans-esophageal echocardiography. In one example, a trans-illumination implementation uses two inserted probes (one illuminating and one detecting) to collect transmission spectra of tissue between the probes. Transmission spectra of skeletal muscle may, in some cases, be obtained through the skin.

In use, end 18 is placed near, or held in contact with, the tissue sample or at a selected position in contact with an organ, for example in contact with cardiac muscle, skeletal muscle or skin. Contact with the sample can be continuous, intermittent or periodic. Sample measurement can be continuous, intermittent or periodic.

Exposure times are typically 50-200 ms, however, exposure times that are greater or shorter are also contemplated. For example, suitable results can be obtained with exposure times of 20, 400, 1000, 2000, or 4000 ms. In one example, the reflected light has penetrated approximately 1 mm into the heart and is a true tissue measurement (not just a surface measurement).

Various elements of those shown in FIG. 1 and FIG. 2 can be coupled by any number of wired or wireless links. A wireless link can rely on a radio frequency transmission using a carrier, an optical link (including fiber elements), or a network connection which can include a variety of wired or wireless connections.

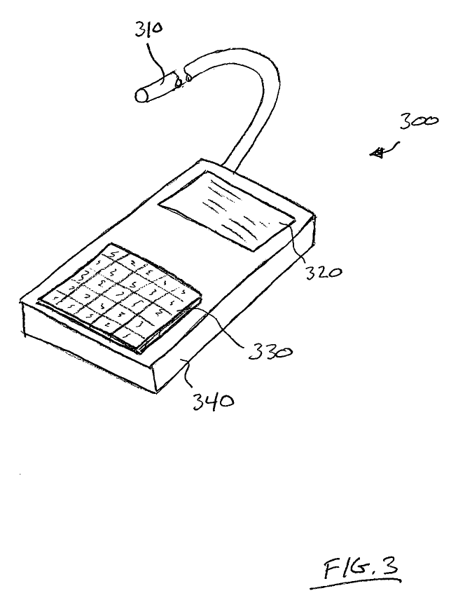

Selected portions of system 100 can be disposed within a portable housing having an internal battery (or other power supply). FIG. 3 shows an example of system 100 configured as a portable device tailored for use as clinical monitoring device 300.

Device 300 includes probe 310 having an optical fiber coupled to a detector and circuitry (not shown) disposed within housing 340. The circuitry includes a battery powered processor and a memory. The memory stores, for example, calibration spectra, the measured data and executable instructions for implementation of an algorithm by the processor. A user interface of one example of device 300 includes a display, a keyboard and a pointing device (such as a mouse or a trackball). Results of the analysis are presented on display 320. A user can control the operation of device 300 using keyboard 330 disposed on a surface of housing 340. Keyboard 330 includes a plurality of user operable switches. Results generated by device 300 can be stored in memory (internal or external to housing 340), displayed on display 320, or printed (using an external printer) or transmitted wirelessly. In one example, housing 340 is portable and includes at least a portion of computer system 23 disposed therein.

Device 300 can include a connector or a wireless transceiver to enable communication with a network or with another device such as a printer or a storage device.

Probe 310, in one example, includes a bifurcated fiber optic element. A light source and a detector are disposed within housing 340 and coupled to probe 310. Device 300 can be tailored to generate data based on transcutaneous illumination of tissue such as a heart or leg muscle. In one example, probe 310 is inserted into a muscle or other tissue.

A memory of device 300 stores a library or database for comparison or interpolation. In one example, the database includes data suitable for use with children, adults and people of different races or ethnicity. Device 300 is configured to be insensitive to stray light or skin pigmentation.

Device 300 can be used for cytochrome measurements of mitochondrial function. In one example, device 300 is configured for myoglobin measurement. As used herein “spectra” includes optical spectra measured by reflectance or transmission.

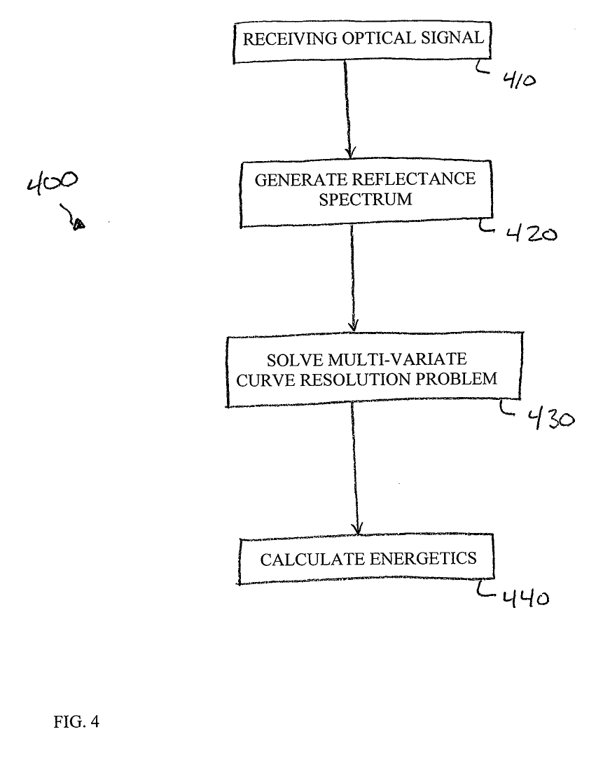

FIG. 4 illustrates method 400 according to one example of the present subject matter. At 410, an optical signal is received from blood-perfused tissue such as a heart, a skeletal muscle, or other biological tissue. At 420, a spectrum is generated for a plurality of wavelengths based on the optical signal. In method 400, a reflectance spectrum is depicted, however, a transmission spectrum can also be used. At 430, a multivariate curve resolution problem is solved using the measured data. Solving the MCR problem can include an iterative method as described elsewhere in this document. At 440, cellular energetics are calculated using the results of MCR analysis. In various examples, the cellular energetics can include assessing mitochondrial characteristics such as oxidation state of cytochrome c and oxidation state of cytochrome oxidase. In one example, more than one characteristic can be determined.

One example of method 400 includes executing an MCR algorithm using, for example, alternating least squares (ALS) in a process referred to as MCR-ALS. The process of MCR-ALS analysis can be summarized as (1) acquiring or generating experimental data matrix; (2) estimating the number of different species and components; (3) estimating initial values; and (4) performing optimization using an iterative process of alternating least squares routine.

One example of method 400 includes executing a statistical multivariate analysis algorithm. In one example, the optical signal is derived from a white light source illuminating a tissue. The optical signal can be received using a fiber optic element.

Component spectra can be obtained from solutions of the pure absorbing species present in tissue—for example, myoglobin, cytochrome oxidase, cytochrome c, and hemoglobin. These can be reflectance spectra from solutions with a light scattering agent added (e.g. 1% Intralipid) or transmission spectra of non-scattering solutions.

Following spectral acquisition in the oxygenated state, excess sodium dithionite can be added to each solution so that spectra of completely deoxygenated or reduced (for the cytochromes) solutions can be obtained. Spectra obtained in the pure state for each absorbing species are then used as the first guess spectra for the MCR algorithm. The accuracy of the first guess spectra is not critical and in one example, the calculation proceeds without using first guess spectra.

### Data Analysis

Multivariate curve resolution can be used to separate and quantify the individual spectral components that are present in observed spectra and in the absence of prior information about the spectral components. MCR-ALS is one example of a routine to implement MCR using an optimization algorithm.

In general, least squares optimizations refer to problems where the objective function includes a sum of squares. In vivo optical spectra can be expressed as a linear combination of the spectra of the individual components, X=CS where X is a matrix describing the in vivo spectra, C is a matrix of the concentrations of the individual components, and S is a matrix of the component spectra. In the context of the present subject matter, ALS refers to estimating matrix C, given an initial starting point for S (the set of solution spectra). Since neither C nor S is known for in vivo spectra, the MCR algorithm includes alternately performing least squares optimizations on matrix C and then on matrix S in each iteration.

The MCR algorithm determines the concentrations of the individual species present in the tissue sample region interrogated. The saturation of hemoglobin (Hbₛₐₜ) and myoglobin (Mbₛₐₜ) is determined by the ratios:

Hbₛₐₜ(%)=100×(Hb_{oxy})/(Hb_{oxy}+Hb_{deoxy})

Mbₛₐₜ(%)=100×(Mb_{oxy})/(Mb_{oxy}+Mb_{deoxy})

Subscripts ‘oxy’ and ‘deoxy’ denote the oxygenated and deoxygenated forms of the proteins, respectively.

The redox state of either cytochrome is defined as the ratio of the oxidized cytochrome to the total cytochrome:

Cytochrome oxidation (%)=100×Cyt_{oxidized}/(Cyt_{oxidized}+Cyt_{reduced})

where Cyt_{oxidized} is the concentration of the oxidized form and Cyt_{reduced} is the concentration of the reduced form of the cytochrome.

In one example, second derivatives of the component spectra and tissue spectra are calculated to reduce the effects of scattering on the spectra and to remove baseline offsets. In one example, discrete derivatives are determined using a second difference algorithm. Computing the second derivative can be viewed as a preprocessing step to MCR. Second derivatives of the spectral data and the first-guess spectra can be computed prior to MCR analysis.

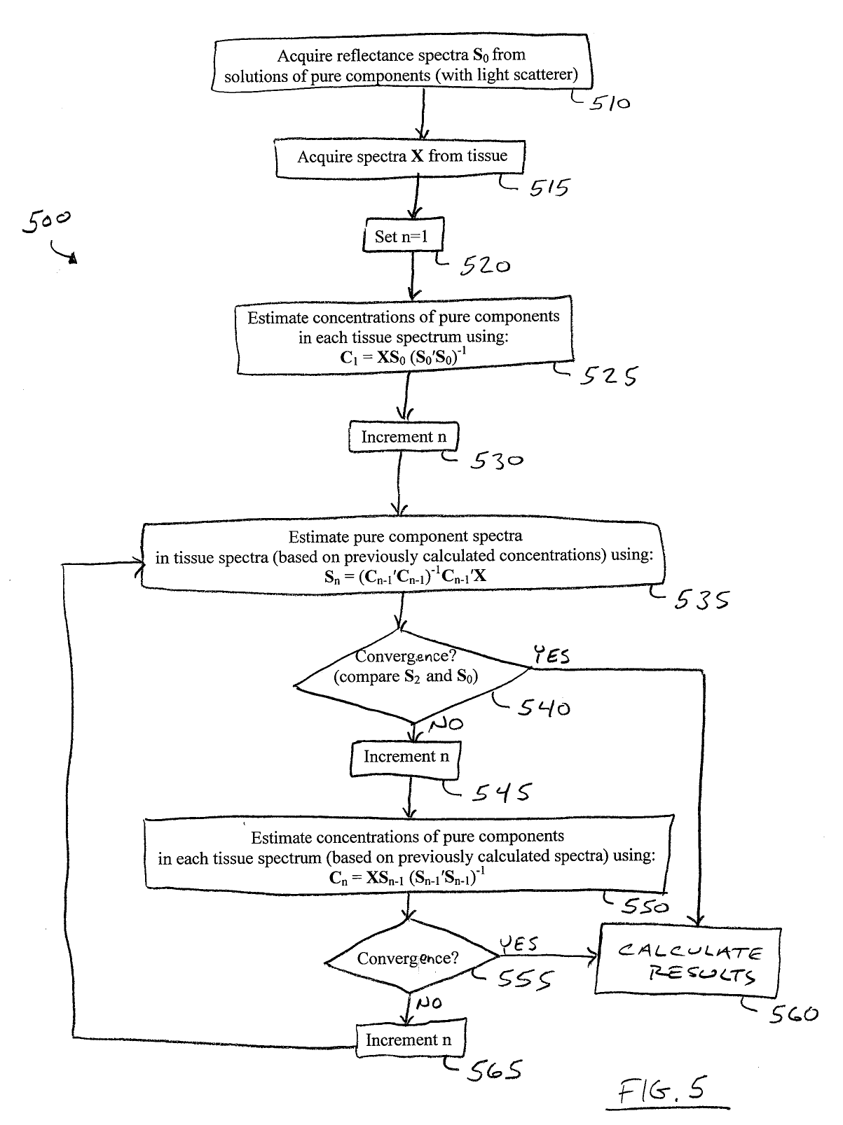

An example of MCR-ALS is illustrated by method 500 shown in FIG. 5. Two sets of spectra are used for each MCR-ALS analysis. The first set of spectra are collected from tissue (X) and the second are collected from solutions of the pure components present in tissue (S₀). The spectra for the pure components can be, for example, collected using beaker samples and stored in a memory of a portable device. At 515, spectra in X are acquired from a human subject or other specimen. Spectra in X can include transcutaneous spectra. At 510, spectra S₀ are reflectance spectra and are used as an initial (or first) guess.

The six solution spectra used to generate S₀ in this example, each include a light scatterer that mimics the scattering found in tissue, and the concentrations of all solution components are known. Uppercase letters in boldface indicate an array of numbers. In X and S₀, the rows of the arrays represent spectra.

In muscle tissue, the main components that absorb light in the visible and near infrared regions are myoglobin, hemoglobin, and the cytochromes. Myoglobin and hemoglobin each have two spectrally distinct states, the oxygen-bound state and the oxygen-unbound state. Cytochrome oxidase is either reduced or oxidized, and these states are also spectrally distinct.

At 520, counter n is set to one.

At 525 (a first stage of the iterative MCR-ALS process), an estimate is made of the concentrations of all pure components present in each tissue spectrum. A linear least squares minimization can be used to determine these concentration estimates. Assume that X and S₀ are linearly related by the concentration matrix C:

X=C₁S₀, (Equation 1)

where the subscript “1” denotes the first iteration (n=1). The least squares estimation is calculated with

C₁=XS₀(S₀′S₀)^{-l}, (Equation 2)

or more generally

Cₙ=XSₙ₋₁(Sₙ₋₁′Sₙ₋₁)⁻¹, (Equation 3)

where ′ indicates matrix transposition and the superscript ⁻¹ indicates matrix inversion.

At 530, counter n is incremented.

At 535, the next iteration stage, the concentrations found in the previous stage (here, C₁) are used to estimate the component spectra found in the tissue. The same general equation that expresses the linearity between X, C, and S is used again, namely,

X=C₁S₂, (Equation 4)

but this time, the least squares estimations of the component spectra are computed by

S₂=(C₁′C₁)⁻¹C₁′X, (Equation 5)

or more generally

Sₙ=(Cₙ₋₁′Cₙ₋₁)⁻¹Cₙ₋₁′X. (Equation 6)

At 540, the iterative process is checked for convergence by quantifying the similarity between spectra in S₂ and S₀. If the difference is larger than a specified threshold, the process continues to 545 where counter n is incremented. If the difference indicates convergence, then processing continues to 560 where results are calculated.

At 550, component concentrations C₃, are estimated by Equation 3, given S₂ and X.

At 555, convergence is again checked and if the difference is below a threshold, then, at 560, the results are calculated. If not sufficiently converged, then at 565, the counter is incremented and processing returns to 535 where pure component spectra are estimated.

Least squares estimates in Equations 3 and 6 are repeated with increasing n values until spectra in S are no longer changing, and the iterative process is stopped.

With alternating least squares estimations of concentrations and component spectra, good estimates of both can be obtained.

After the iterative solution has converged, then at 560, myoglobin saturation (mb sat), hemoglobin saturation (hb sat), and cytochrome oxidation (cyt ox) are calculated from concentrations in the final Cₙ matrix as follows:

Hbₛₐₜ (%)=100×(Hb_{oxy})/(Hb_{oxy}+Hb_{deoxy})

Mbₛₐₜ (%)=100×(Mb_{oxy})/(Mb_{oxy}+Mb_{deoxy})

Cytochrome oxidation (%)=100×Cyt_{oxidized}/(Cyt_{oxidized}+Cyt_{reduced})

The MCR calculation can be performed by methods other than ALS, including for example Positive Matrix Factorization, Independent Component Analysis, Maximum Autocorrelative Factors, and Evolving Factor Analysis. These algorithms generally do not use linear least squares criteria, and may not be iterative in nature. More generally, MCR includes a class of algorithms that uses an observed signal to estimate the chemical and physical characteristics of the contributing components and the quantity of components in each sample.

As described here, the concentrations and component spectra can be determined using MCR. In addition to MCR, other members of a class of algorithms called regression methods include classical (ordinary) least squares, partial least squares, and multiple linear regression, any of which can be adapted for use with the present subject matter.

### Surgical Example

In one example, optical spectra from the myocardium are acquired during various stages of a surgical procedure, or continuously, such as prior to initiation of cardiac bypass, during the time period on bypass (cardioplegia administered), and during the transition off of bypass (return to blood perfusion of the heart).

Spectra can be obtained continuously over an extended time period such as days, weeks or months. An indwelling probe can be used and data can be stored (either locally or remotely) and conveyed to a computer for analysis.

During the period of bypass, when the heart receives no new oxygen supply, spectra are collected at multiple time points, or continuously, in order to track intracellular oxygenation and mitochondrial cytochrome redox.

The optical probe is placed on the surface of the heart and held there manually while spectra are obtained. The probe is placed in the same position on the heart each time. In one example, a holder or attachment device is used to maintain the probe in a particular place.

The optical spectra acquired during open-heart surgery are analyzed by MCR to obtain measurements of myoglobin and hemoglobin saturations and cytochrome c and cytochrome oxidase redox. Intracellular oxygenation (as determined by myoglobin saturation), and energy status of the cells (as reflected by cytochrome redox), before, during, and after bypass can be correlated with outcome variables.

In one example, optical spectra are acquired from an optical probe placed on skeletal muscle during a surgical procedure. The optical spectra acquired during surgery are analyzed by MCR to obtain measurements of one or more of myoglobin and hemoglobin saturations and cytochrome c and cytochrome oxidase redox. Intracellular oxygenation (as determined by myoglobin saturation), and energy status of the cells (as reflected by cytochrome redox), before, during, and after surgery can be correlated with outcome variables.

### Skeletal Muscle Example

In one example, intracellular oxygenation and cytochrome redox measurements in skeletal muscle are made through the skin. The signal amplitude collected from skeletal muscle is generally lower than in the example of cardiac surgery. In skeletal muscle measurements, light traverses the skin and other tissue layers while traveling to and from the muscle below. The present subject matter provides a method of measurement of cytochrome redox state, hemoglobin saturation and/or myoglobin saturation as an assessment of tissue intracellular energy status.

The present subject matter enables distinguishing cytochrome oxidase from myoglobin and hemoglobin in a blood-perfused muscle where the cytochrome concentrations are much lower than hemoglobin and myoglobin concentrations.

The present subject matter utilizes different wavelength regions with the calibration spectra tailored (or trained) to discern the cytochrome information from the sample.

The present subject matter includes illuminating a sample using an optical probe and analysis of the returned signal. The returned signal, or spectrum, from the muscle is analyzed to determine what part of that signal is derived from oxidized or reduced cytochrome, yielding redox state.

In one example, different solutions are formed with distinct solutions including one of the cytochromes, one solution including myoglobin and one solution including hemoglobin. The spectra of the solutions (either in the oxidized form or the reduced form or with oxygen bound or without oxygen bound) is stored. In one example, the solutions include Intralipid (a lipid emulsion) to increase turbidity and appear more like muscle tissue. The Intralipid (or other light scattering agent) is added in a concentration to approximate the light scattering property of tissue. In another example, solutions do not include a scatterer, and the diffusion equation is used to add the effects of scattering to the spectra.

The comparison of the measured spectra and the calibration spectra involves multi-wavelength analysis. This includes analysis and comparison of a range of wavelengths. In one example, multivariate curve resolution analysis, or other multivariate analysis, is used to analyze the tissue spectral data. Various spectral analysis methods can be used with the present subject matter. Exemplary methods include multi-wavelength or multivariate methods.

The present subject matter provides quantitative levels of oxygen saturation and redox states of the cytochromes. The present subject matter uses a range of wavelengths (for example, 100-150 wavelengths or points) and multivariate analysis. For example, myoglobin and hemoglobin have similar absorption spectra and their spectra are so similar that at a particular wavelength, absorbance will trend in the same direction.

Various ranges of wavelength regions can be used. For example, in the range of 500-700 nanometers, the detector includes a spectrometer having 200 pixels (photodetectors) in that range. In one example, the spectrum is generated for a range of wavelengths including those greater than 700 nm or less than 500 nm.

As to spectral analysis, the present subject matter relates to a single wavelength range or several wavelength ranges. As to a spectrometer, the present subject matter relates to measurement of a plurality of wavelengths.

The MCR algorithm can be combined with partial least squares (PLS) or other algorithms. In PLS, a mitochondrial characteristic of the tissue is calculated using the reflectance spectrum and a calibration set. The calibration set includes data corresponding to in vitro solutions of at least one of myoglobin, hemoglobin, cytochrome c, and cytochrome oxidase. A partial least squares algorithm is trained to a particular cytochrome in calculating the characteristic.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

A method example described herein can be machine or computer-implemented at least in part. Some examples can include a computer-readable medium or machine-readable medium encoded with instructions operable to configure an electronic device to perform a method as described in the above examples. An implementation of such a method can include code, such as microcode, assembly language code, a higher-level language code, or the like. Such code can include computer readable instructions for performing at least one of various methods. The code may form a portion of a computer program product. Further, the code may be tangibly stored on one or more volatile or non-volatile computer-readable media during execution or at other times. These computer-readable media may include, but are not limited to, a hard disk, a removable magnetic disk, a removable optical disk (e.g., a compact disk and a digital video disk), a magnetic cassette, a memory card or a stick, a random access memory (RAM), a read only memory (ROM), and the like.

### CONCLUSION

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
a data source configured to provide data corresponding to optical spectra for a tissue;
a data analyzer coupled to the source, the data analyzer configured to generate an estimate of a cellular energetic parameter using a multivariate curve resolution algorithm and using the data from the data source; and
an output device coupled to the data analyzer and configured to render an output based on the parameter.

2. The system of claim 1 wherein the data source includes a probe having an optical transmitter and an optical detector and wherein the optical detector is configured to provide spectral data using light from the optical transmitter.

3. The system of claim 2 wherein the probe provides data corresponding to reflectance spectra.

4. The system of claim 2 wherein the probe provides data corresponding to transmission spectra.

5. The system of claim 2 wherein the probe is configured for external use.

6. The system of claim 2 wherein the probe is configured for internal use.

7. The system of claim 1 wherein the data analyzer is configured to determine a concentration, saturation, oxygenation, oxidation, or a content.

8. The system of claim 1 wherein the data analyzer includes a processor having instructions for executing an iterative process.

9. The system of claim 8 wherein the iterative process includes an alternating least squares process.

10. A method comprising:
receiving data corresponding to optical spectra from a tissue sample;
alternately estimating concentrations of pure components for each tissue spectrum and estimating pure component spectra using an iterative process;
monitoring for convergence between two iterations; and
upon detecting convergence, calculating a cellular energetic parameter based on the estimated concentrations.

11. The method of claim 10 wherein estimating the pure component spectra includes determining a concentration.

12. The method of claim 10 wherein receiving data includes receiving data from an optical probe.

13. The method of claim 10 wherein receiving data includes receiving data corresponding to at least one of visible light and near infrared light.

14. The method of claim 10 wherein estimating the concentrations includes accessing stored information.

15. The method of claim 10 wherein alternately estimating includes executing a multivariate curve resolution algorithm.

16. The method of claim 15 wherein executing the multivariate curve resolution algorithm includes using alternating least squares optimization.

17. The method of claim 10 wherein monitoring for convergence includes comparing a stored value and a difference between two pure component spectra.

18. The method of claim 10 wherein calculating the cellular energetic parameter includes calculating at least one of myoglobin saturation, hemoglobin saturation, and cytochrome oxidation.

19. A device comprising:
a portable housing;
a user interface coupled to the portable housing, the interface having at least one user operable switch and an output device;
a processor disposed in the housing and coupled to the user interface; and
a probe coupled to the processor, the probe having an illumination source and an optical receiver element, the optical receiver element configured to generate data using a specimen tissue and using the illumination source; and
wherein the processor is configured to execute an algorithm for implementing multivariate curve resolution analysis, the processor having access to a memory with instructions stored thereon for performing the analysis using the data and further wherein the processor is configured to generate at least one cellular energetic parameter using a result from the analysis.

20. The device of claim 19 wherein the user interface includes at least one of a keyboard and a pointing device and further wherein the output device includes a display.

21. The device of claim 19 wherein the processor is configured to implement multivariate curve resolution using alternating least squares optimization.

22. The device of claim 19 further including a battery coupled to the processor.

23. The device of claim 19 wherein the probe is configured to generate data corresponding to at least one of visible light and near infrared light.
